(19) United States
(12) Patent Application Publication
(10) Pub. No.: US 2011/0160075 A1
(43) Pub. Date: Jun. 30, 2011

(54) Diagnostic assay for orientia tsutsugamushi by detection of responsive gene expression

(76) Inventors: Wei-Mei Ching, Bethesda, MD (US); Xuan Li, Silver Spring, MD (US)

(21) Appl. No.: 12/927,807

(22) Filed: Nov. 24, 2010

Related U.S. Application Data
(63) Continuation-in-part of application No. 11/357,462, filed on Feb. 9, 2006.

(51) Int. Cl. C40B 30/04; C12Q 1/68; G01N 33/53

(52) U.S. Cl. 506/9; 435/6.12; 435/7.92

(57) ABSTRACT

The inventive subject matter relates to a method for the diagnosis of Orientia tsutsugamushi infection by measuring the increased or decreased expression of specific human genes following infection by microarray or polymerase chain reaction analysis. The method employs the creation of gene modulation profiles in patients suspected to be infected with O. tsutsugamushi and comparing the profiles with a pre-determined profile of genes known to modulate in response to O. tsutsugamushi exposure and infection. The method permits the early detection of O. tsutsugamushi infection and diagnosis of scrub typhus earlier than currently available methods. The method also permits mid-course monitoring of disease progression with greater detail than currently available methods.

### CROSS-REFERENCE TO RELATED APPLICATIONS

This application is a continuation-in-part application of application Ser. No. 11/357,462.

### BACKGROUND OF INVENTION

1. Field of Invention

The inventive subject matter relates to a method of diagnosing Rickettsial diseases by analysis of modulation of host gene expression. The method contemplates the use of microarray technology for the detection and analysis of gene up or down regulation in response to bacterial infection.

2. Description of Related Art

The disease scrub typhus, caused by the Gram negative bacteria Orientia (formerly Rickettsia) tsutsugamushi is one of the most common rickettsial diseases and can cause up to 35% mortality if left untreated (1, 2). The bacterial pathogen accounts for up to 23% of all febrile episodes in endemic areas of the Asia-Pacific region. Geographic distribution of the disease occurs principally within an area of about 13 million square kilometers and includes Pakistan, India and Nepal in the west to Japan in the east and from southeastern Siberia, China and Korea in the north to Indonesia, Philippines, northern Australia and the intervening Pacific islands in the south. During World War II, more than 5,000 cases of scrub typhus were reported among U.S. troops and 30,000 cases for Japanese troops. Scrub typhus ranked only behind malaria as the most important arthropod borne infectious disease. More recently, scrub typhus was the second leading cause of fevers of unknown origin among U.S. personnel during the Vietnam conflict.

Because of the relatively high mortality rate in untreated patients, the rising prevalence of drug resistant strains, and the lack of vaccines against the organism, early detection of exposure and infection is becoming increasingly important. For this reason, simple and accurate methods are important for early detection and effective treatment of the disease. However, despite the global public health importance of scrub typhus, currently available diagnostic methods are inadequate. Diagnosis of scrub typhus is generally based on the clinical presentation and history of the patient. Because of similarities in symptomatology, however, differentiation of scrub typhus from other febrile diseases, such as leptospirosis, murine typhus, malaria, dengue fever and viral hemorrhagic fevers, is often difficult especially early after infection.

In order to overcome the short-comings in scrub typhus diagnosis, significant research effort has been devoted to developing accurate laboratory diagnostic methods for scrub typhus. The currently available assays are typically seriologically-based and include indirect-fluorescence assay (IFA), indirect immunoperocxidase assay (IIP), enzyme-linked immunosorbent assay (ELISA) and dot blot assays. These assays, however all suffer from the requirement of requiring the availability of antigen which typically entails growing rickettsiae grown in host cells or preparing extracts of purified bacteria as well as the availability of antibody in patient sera (3-10). Additionally, the assay methods are time consuming to perform and offer limited insight into serotypes not represented by the panel of available antigen.

A problematic hurdle in the design of sensitive and accurate diagnostic assays is ensuring the assay's effectiveness early after infection. In currently available and employed antibody-based assays, sensitivity requires a suitable number of bacteria in tissue samples. Typically, adequate levels of bacterial load to meet the required threshold are not found, especially early after an infection. Likewise, detection of seroconversion is also not an effective diagnositic method early after exposure and infection since no detectable, specific antibody would be present.

Other confounding issues in designing suitable assays include the fact that Orientia strains exhibit significant antigenic differences thereby complicating assay antigen selection for use in available scrub typhus serodiagnostic procedures. For example, the major outer membrane protein (vOmp) of O. tsutsugamushi is an important serodiagnostic antigen but varies from 53-63 kDa even among isolates from the same country (11). Furthermore, both unique and cross-reactive domains exist in different homologs that potentially necessitating the use of multiple strains in scrub typhus diagnostic test design. Additionally, the list of scrub typhus serotypes is incomplete.

Polymerase chain reaction (PCR) amplification of O. tsutsugamushi genes has been demonstrated to be a reliable diagnostic method for scrub typhus (12, 13). PCR permits the rapid identification of distinct genetypes that are associated with Orienta serotypes (12, 14-18). However, despite the advantages of PCR, significant disadvantages include the requirement for sophisticated instrumentation and labile reagents to conduct the assays that are often not available in rural medical facilities. Additionally, PCR procedures are highly susceptible to false positive results due to inadvertent carry-over of nucleic acid material. This is particularly prevalent in field settings or in facilities that are not fully equipped to conduct PCR laboratory procedures.

A solution to the paucity of early diagnostic methods is to monitor the expression of host response genes in response to infection. Early after exposure to an infectious organism, host responsiveness to infection is manifested by modulation of specific gene expression. Some genes are differentially expressed very early after infection thus permitting the construction of unique gene expression profiles that are exhibited early after infection of human cells, such as peripheral blood mononuclear cells (PBMC). The patterns or profiles of gene expression would thus enable the differentiation of exposure by pathogens and toxins, including Bacillus anthracis, Yersinia pestis, Brucella melitensis, botulinum toxin, staphylococcal exotoxins A and B (SEB, SEA), lipopolysaccharide (LPS), cholera toxin, Venezuelan equine encephalitis virus (19). Furthermore, it has been previously shown that specific human genes modulate up or down in response to bacterial infection (20).

Semi-quantitative reverse transcriptase polymerase chain reaction (RT-PCR) is capable of sensitively measuring changes in gene expression from collected host cell RNA. By designing primer sets specific to a limited number of genes, known to have altered expression following infection, molecular-based assays can be devised to diagnosis and monitor infection early after infection by direct assessment of gene modulation.

Although measurement of changes in gene expression by RT-PCR is a valuable diagnostic strategy, the method suffers from the disadvantages associated with PCR in that it is often not suitable for high-throughput screening of large numbers of genes. A more convenient method of measuring gene expression changes is by hybridizing amplified RNA onto cDNA microarrays containing large numbers of double-stranded sequences of important host genes. A number of computer programs are available to accurately analyze and transform the ensuing gene expression data into useful and reproducible gene expression profiles.

Microarrays are well suited for high-throughput detection of thousands of differentially expressed genes in a single experiment (21). The method allows for the characterization of the cascade of cellular signaling and concomitant interrelated host gene expression profiles following infection by specific pathogens or toxins (22, 23). Therefore, data from cDNA microarrays provides the ability to quickly and accurately assess and monitor the changes in gene expression profiles specific to infection by specific pathogenic organisms. Microarrays can also be used to evaluate genomic differences between virulent and nonvirulent strains of a species (24).

Therefore, in order to improve early diagnosis of scrub typhus, an aspect of this invention is the diagnosis of O. tsutsugamushi early after exposure and infection by the measurement of specific host gene expression profile. The invention, therefore, will give diagnosticians the ability to diagnosis O. tsutsugamushi days or weeks earlier than previously possible with a concomitantly greater likelihood of accuracy in disease etiology. Additionally, the care provider will be able to accurately monitor the course of the disease, thereby facilitating the selection of effective drug regimens.

### SUMMARY OF INVENTION

Current methods for the detection and diagnosis of scrub typhus, caused by the rickettsial organisms Orientia tsutsugamushi early after infection are inadequate. An object of this invention is a method for diagnosis of O. tsutsugamushi early after exposure and infection to the organism and the monitoring of disease course by the modulation of expression of specific host cell genes.

A further object of the invention is the diagnosis of O. tsutsugamushi by polymerase chain reaction with low background due to amplification of contaminating DNA.

### BRIEF DESCRIPTION OF DRAWINGS

FIG. 1 Verification of PBMC infection by O. tsutsugamushi and quality analysis of RNA extracted from uninfected and infected PBMC. Panel (A) shows the PCR amplicon of GroEL gene from the O. tsutsugamushi genome. DNA from each sample was used as template in PCR to generate 548 bp amplicon representing a segment of the conserved GroELS gene in O. tsutsugamushi. Pure genomic DNA extracted from O. tsutsugamushi Karp strain was used as positive control (lane 2). Uninfected samples (lane 3-7) and infected samples (lanes 8-11) obtained 1, 4, 8 and 18 hrs post infection from one of the donors were used for the experiment. Lane 1 is 1 kb DNA ladder standard. Panel B shows the quality of RNA examined using Agilent Bioanalyzer 2100. The 28S and 18S rRNA were observed as two distinct bands in most of the samples. Lanes 1-13 are duplicated samples from different time points from one donor.

FIG. 2. Fluorogenic real time PCR determination of NM_{—}001547 (interferon-induced protein with tetratricopeptide repeats 2 (IFIT2)) and NM_{—}006187 (2′-5′˜oligoadenylate synthetase 3, 100 kDa (OAS3)). Uninfected and infected samples from donor 2 were used for analysis. Panels A through D shows expression of interferon induced protein using mRNA from O. tsutsugamushi infected and uninfected leukocytes by real-time polymerase chain reaction. Panels E through H shows the expression of 2′-5′ oligoadenylate synthetase 3 using mRNA from O. tsutsugamushi infected and uninfected leukocytes by real-time polymerase chain reaction. Panels A and E show two independently run standard curves using different amounts of 18S rRNA. Panel B and F show the difference of each gene between uninfected and infected samples 4 hrs post infection. Panel C and G show the difference of each gene between control and infected samples

### DETAILED DESCRIPTION OF PREFERRED EMBODIMENTS

Diagnosis of the disease scrub typhus caused by O. tsutsugamushi early after exposure of individuals to the bacteria is difficult due to a lack of available assay methods. Current methods for the diagnosis of scrub typhus rely on detection of serum conversion, which is not possible until significant time has elapsed after exposure or the direct detection of the organism which requires a considerable incubation period following exposure.

Analysis of human gene expression profiles has become an increasingly important mode of predicting disease onset and for monitoring disease progression. Following exposure to external insults, such as infectious organisms or toxins, some cellular genes are modulated to increase or decrease expression. Specific cell perturbations can result in precise gene modulation profiles that are predictive for a specific external insult. The current invention capitalizes on this phenomenon by monitoring gene expression early after exposure of human cells to O. tsutsugamushi by measuring mRNA encoding the gene product or by measuring the genes protein product itself. Analysis of the gene modulation profile of cells is highly predictive of prior exposure and infection with O. tsutsugamushi. Therefore, an aspect of the invention is the detection and measurement of changes in gene expression following exposure and infection by Orientia tsutsugamushi.

Analysis of human gene expression can therefore be a predictor of infection by specific microorganisms. The general approach, therefore, of evaluating changes in human gene expression can be utilized as an effective diagnostic tool very early after infection, when other currently available methods are not effective. The approach can be used alone or in tandem with other methods, therefore, to follow progression of the disease state through treatment.

Diagnosis of infection is operationally carried out by initially measuring changes in gene expression in response to infection. Any semi-quantitatively or quantitative procedure can be used to measure changes in expression. A number of methods can be used to measure gene expression. Gene expression profiles can be measured by antibody-based methods, such as enzyme-linked immunosorbent assay (ELISA). In ELISA, a specific quantity of extracted cell protein is immobilized which is then exposed to antibody specific for genes suspected of modulation. The expression of the specific genes are normalized to the expression of a house-keeping gene. Antibody-based assays, however, suffer from the inherent requirement of antibody to selected antigens of interest and time-intensity required to conduct the assay. Therefore, alternative approaches include molecular assay methods.

Measuring changes in gene expression by reverse transcriptase polymerase (RT-PCR) chain reaction is best conducted by constructing primer sets containing at least one of the primers to the mRNA splice site. This aspect of the invention significantly increases specificity and therefore reliability of diagnosis by reducing the amplification of contaminating DNA.

Alternatively, or in addition to RT-PCR, labeled cDNA copies of mRNA from the infected human cells can be exposed to complimentary DNA copies of specific genes attached to glass microarray chips and the bound cDNA quantitated. Use of microarrays permits the convenient analysis of large numbers of genes in a single experiment. RT-PCR can also be used, in conjunction with microarray analysis, to either confirm results or to more accurately determine the relative degree of modulation of target genes. Evaluation of gene modulation profiles is conducted by computer program analysis.

Any semi-quantitatively or quantitative procedure can be used that accurately measures changes in host cell gene expression following bacterial exposure and infection. Regardless of the specific method used, the general approach in all methods employs the following steps:
a. obtaining leukocytes from blood samples from patients potentially exposed to O. tsutsugamushi;
b. extracting total RNA or protein from the leukocytes;
c. measuring gene products of a panel of important host genes by molecular, antibody-based or other methods;
d. normalizing the expression of the important host genes in the potentially infected cells to that in uninfected cells;
e. analyzing the pattern or profile of gene modulation by computer program.

Based on the gene modulation profile, a diagnosis early after exposure and infection is made by comparing the profile detected with that associated with the profile associated with O. tsutsugamushi infection. Since this method permits diagnosis much earlier after infection than other available assay methods, early, and presumably more efficacious, antibiotic treatment can be instituted. Additionally, regular re-evaluation of expressed genes during disease progression permits real-time evaluation of the effectiveness of the drug treatment regimen and modification of treatment methods, if needed. To more clearly describe the invention, the following examples are given.

### Example 1

### Detection of Gene Expression in the in PBLs by Hybridization of Gene Products to Microarray Chips

Peripheral blood lymphocyte (PBL) were utilized as the source of RNA in order to examine the gene expression modulation in response to infection with O. tsutsugamushi. Other cell types, however could be used including purified peripheral blood mononuclear cells or subpopulations such as T-cells, B-cells and macrophages.

Preparation of Orientia tsutsugamushi for infection. The 1 mL seed inoculum stored at −80° C. was thawed at 37° C. and mixed well with 19 mL of brain heart infusion (BHI). Confluent L929 cells in T162 were infected with 2 mL of BHI-Orientia suspension and placed on a rocker platform for 60 minutes with rotation at 90 degrees each 15 minutes to ensure the inoculum was dispersed uniformly over all the cells. The flasks were incubated at 35° C. with 5% CO₂ in M-199 with Earles salts, 5% fetal bovine serum, 2 mM L-glutamine, and 5% tryptose phosphate broth (TPB). The infected L929 cells were harvested 5-8 days post infection (when 30-50% cells deteriorated). The medium was replaced with 10 mL of K36 (16.5 mM KH2P04, 33.3 mM K2HP04, 100 mM KCl and 15.5 mM NaCl, pH 7.2-7.3) buffer solution. Sterile glass beads (5 mm) were added to the flask which was rocked gently to slough the L929 cells from the surface until no cells were attached. The cell suspension was centrifuged at 8,000 rpm in a Sorvall RC-5C centrifuge at 4° C. for 30 minutes. The pellet was resuspended in 5 mL filtered SRM (4.9 mM L-glutamine, 3.6 mM KH2P04, 7.1 mM K2HP04, 218 mM sucrose, 1% Renografin 76 and 5 mM MgCl2) and aliquoted into 2 mL cryovials and stored at −80° C. until use. A slide smear and control for sterility were done to ensure the quality of preparation.

Preparation of PMBCs. Blood from three volunteers was collected into CPT tubes on three different days. Each blood sample was mixed and centrifuged at 1500×g in a swinging-bucket rotor at room temp for 45 min. After centrifugation, the top layer of yellowish plasma was aspirated and discarded. The whitish mononuclear cell layer was aspirated and transferred into 50 mL tubes. Mononuclear cells were washed with PBS twice by centrifugation at 180×g at room temperature for 10 minutes. Cells were resuspended in appropriate volume of PBS (30 mL of PBS for 500 mL blood). The cells were counted using a hemacytometer. An equal number of cells from each individual were used for each time point with or without infection.

Infection of PMBCs with O. tsutsugamushi. PMBCs in PBS were centrifuged at 1,200 rpm for 10 minutes and resuspended with growth medium (GM, RPMI with 7.5% human serum and IX L-glutamine) to obtain 6×106 cells/mL. A vial of O. tsutsugamushi was thawed and added to PMBCs at multiplicity of infection (MOI) of 100 and gently rotated (10 rpm) at 35° C. for 45 minutes. For the uninfected control samples, cells were incubated under the same condition without O. tsutsugamushi. After incubation, PBS was added to the O. tsutsugamushi infected PBMC and centrifuged at 2,000 rpm in a Sorvall RC-5C centrifuge for 5 minutes to wash away any uninternalized Orientia. This step was repeated once and the pellet of PMBCs was resuspended in GM. Eight mL of infected and uninfected PMBCs was added into each well in a 4-well plate containing GM. A total of 2 plates (8 wells) were used for each of the uninfected and infected groups. These plates were left in an incubator after infection.

Incubation of infected cells and RNA extraction at indicated times. Cells were incubated at 5% CO2, 95% humidity and 35° C. for additional 1, 4, 8 and 18 hrs post-infection. At each indicated time, both uninfected and infected PMBCs were removed, centrifuged at 2,000 rpm in a Sorvall RC-5C centrifuge for 5 minutes, and the pellet was resuspended in 1.5 mL Trizol (Invitrogen, CA) for RNA extraction which was performed as described by the manufacturer. The purified RNA was subjected to both quantitative and qualitative analyses with Agilent 2100 Bioanalyzer (Agilent Technologies, Calif.). Only those samples with good quality RNA were used for microarray analysis. High quality RNA with A₂₆₀/A₂₈₀ ratio greater than 1.9 and enough quantity was obtained for microarray studies. Total DNA was also extracted after RNA extraction by Trizol according to the instruction provided by the manufacturer. Extracted DNA (with A₂₆₀/A₂₈₀ ratio greater than 1.8) was used as template in PCR with a primer set specific for the GroELS gene [26] of O. tsutsugamushi to confirm infection of PBMC by Orientia.

DNA microarray Preparation and Image analysis. The cloned gene library for printing microarray slides was obtained from Research Genetics (Invitrogen, Carlsbad, Calif.). The slides contained 7,489 genes, including 7,019 known genes, 249 unknown genes, 110 expressed sequence tagged genes (ESTs), and 111 positive and negative control genes in replicates. Superamine coated Telechem slides (Telechem Inc., OR) were used for printing the cDNA clones using 12×4 pin format, on a Virtek chip writer professional microarrayer at KemTek, Inc, Md. The printed slides underwent UV cross-linking, followed by succinic anhydride treatment. The MICROMAX™ Tyramide Signal Amplification (TSA)™ Labeling and Detection Kit (PerkinElmer, Inc., MA) was used as recommended by the manufacturer to determine relative gene expression of the collected samples. Human reference RNA was obtained from Stratagene and was used on every slide as the array control to check overall sensitivity of array printing and to monitor reverse transcription, labeling and hybridization efficiencies. Sample hybridization was carried out at 65° C. for sixteen hours. A laser detection system was used (GenePix 4000b, Axon Instruments, Calif.) to scan the finished slides. The intensity of the scanned images was digitalized through Genepix 4.0 software (Axon Inc., CA) [27].

Data Analysis for Microarray. Data filtering and statistical analysis were carried out using GENESPRING® 7.0 (Agilent Technologies, Santa Clara, Calif.). Local background was subtracted from individual spot intensity. Genes that failed this ‘background check’ in any of the given experiments were eliminated from further analysis. Next, each chip was subjected to intra-chip normalization (LOWESS). The genes that varied most between infected and uninfected sample sets were selected via ANOVA t-test analysis followed by Benjamin correction in order to reduce false discovery rate of less than 5%. A two dimensional hierarchal clustering calculation using Pearson correlation around zero was also performed.

If the PBLs had been obtained from presenting patients, early treatment, prior to that capable using currently available methods, can be initiated. Diagnosis is made by comparing and contrasting the gene modulation profile of the obtained PBLs with the expected gene induction following infection with O. tsutsugamushi.

Follow-up, confirmatory diagnostic assays, such as RT-PCR or ELISA and other antibody-based assays for the detection of bacterial antigen, can be undertaken in order to give further assurance of infection and strain identification. Furthermore, additional assays, during the course of the disease, by microarray analysis or by other traditional diagnostic methods using fresh PBLs, can be undertaken to monitor the disease progression and effectiveness of treatment.

Using cDNA arrays and various bioinformatics tools, gene expression profiles induced by intracellular O. tsutsugamushi in human PBMC were measured at early stages of infection. Expression ratios of genes in the cDNA arrays were determined by comparing the levels of mRNA in Orientia infected cells vs. uninfected cells at each time point. The results were represented as the average of blood samples analyzed from three separate donors.

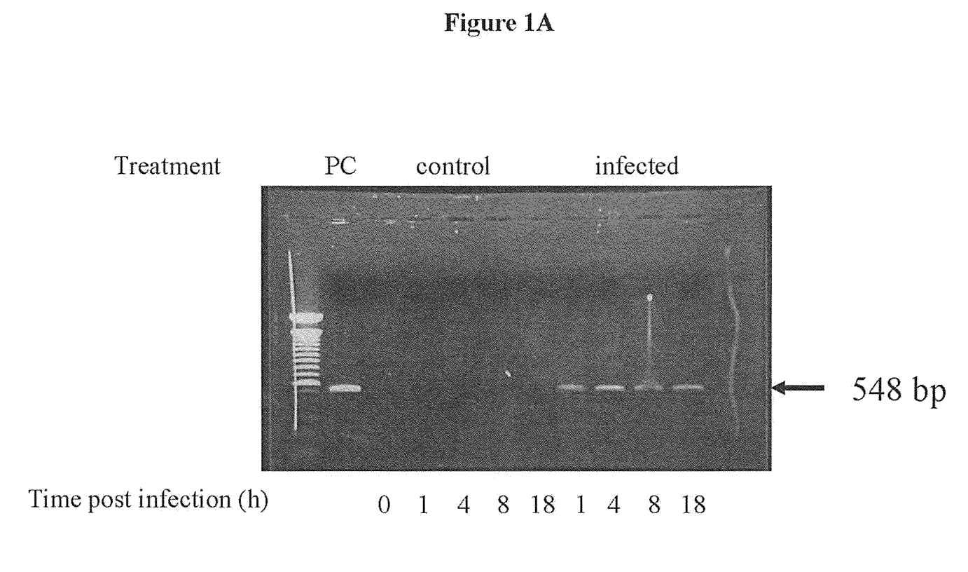

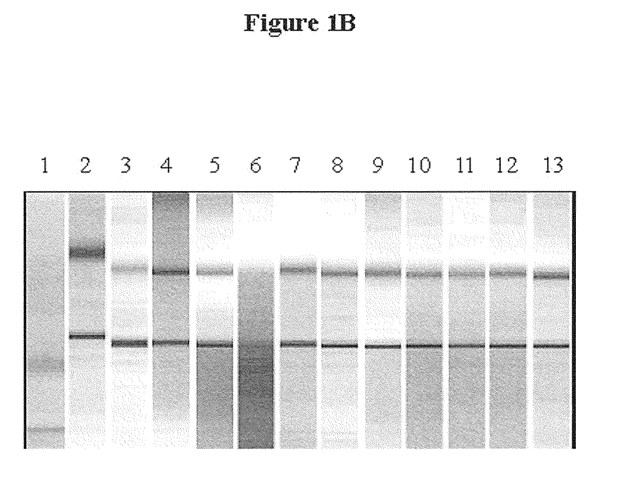

In this example, the infection of PBMCs with O. tsutsugamushi was confirmed by Giemsa staining (data not shown). Furthermore, the presence of Orientia DNA in infected samples was demonstrated by PCR using primers for the groELS. The results is shown in FIG. 1. (A), clearly indicates that the O. tsutsugamushi DNA was only detectable in the infected samples (amplicon of 548 bp long as indicated) at all time points but not in the uninfected samples confirming that PBMCs were infected by O. tsutsugamushi. The quality of the extracted RNA was evaluated by the integrity of the 18S and 28S rRNA (28). As shown in FIG. 1 (B), it was clear that the majority of the RNA preparations was of high quality and could be used in the downstream microarray application. The apparent differences in mobility were inspected by using analysis software provided by the manufacturer (Agilent Technologies).

Gene expression profiles of 658 genes or 9% of the genes on the microarray showed more than 2-fold change in expression during Orientia infection. These genes belong to many different functional categories such as cytokines, transcription factors, kinases and phosphatases, genes involved in hemostasis, coagulation and apoptosis. Up and down regulations of a variety of different classes of gene families were observed in a time dependent manner. The 2-fold change of expression was selected arbitrarily as the cutoff for further studies due to the excessive number of genes. There are 432 genes with statistically significant changes based on ANOVA t-test (p<0.05).

### Example 2

### Analysis of Gene Modulation by Polymerase Chain Reaction

Gene modulation can be determined by quantitative reverse transcriptase polymerase chain reaction (RT-PCR). RT-PCR analysis can also be used alone or in tandem with other methods, such as microarray analysis, in order to confirm the results obtained by that method.

In this embodiment, both semi-quantitative PCR and quantitative PCR analyses were performed to confirm the list of genes of which expression level was affected by O. tsutsugamushi infection. Following experiment in example 1, the first strand cDNA synthesis reaction was carried out in a 100 μl reaction volume containing 15 μl of the total RNA from each sample, previously denatured at 70° C. for 5 min and cooled on ice for 3 min, in the presence of 2 μl oligo dT, dNTPs, DTT, Superscript II RT (Invitrogen) and RT buffer following the manufacturer's instruction. The reaction mixtures were incubated at 42° C. for 50 min, then at 70° C. for 10 min. PCRs were performed using the gene specific primers for GAPDH (housekeeping gene, glyceraldehyde 3-phosphate dehydrogenase) (Forward: 5′-ACTGGCGTCTTC ACCACCATG-3; Reverse: 5′-CCACCTGGTGCTCAG TGTAG-3′) in the presence of a 1:5 fold serial dilutions of the newly synthesized cDNAs. The PCR mixture was incubated at 94° C. for 3 min followed by 30 cycles of a 3-step amplification at 94° C. for 30 sec, 62° C. for 30 sec, and 72° C. for 1.5 min. PCR products (size 551 bp) were separated by electrophoresis on a 1% agarose gel. The unsaturated band from the gel image data was selected from each cDNA sample and semi-quantitation of each sample was performed by using Gel analyzing software, GelPic Analyzer 1.2 (GeneHarbor Inc. Gaithersburg, Md.). After background subtraction, the normalization factor of the cDNA template was determined based on the intensity of the band corresponding to GAPDH. The normalization factor was used to semi-quantitate the relative amounts of gene expression based on the amount of GAPDH as the internal standard.

After production of a cDNA copy of the RNA by reverse transcriptase, primers to selected targets are used to amplify specific target genes sequences. This was performed in order to eliminate the possibility of PCR amplification due to contaminated DNA in the RNA preparations and to allow for the detection of specific amplicons from matured mRNA. Primer sets are designed such that at least one primer member of a primer set is complementary to the sequence encoding the splice site of the target mRNA. Targeting of primer sequences complementary to splice junctions ensures that amplification of sequences will not occur using genomic DNA as template. Thus background amplification due to amplification of remaining DNA, despite treatment of RNA with DNase will be minimized.

a. Semi-Quantitative PCR

Specific primers that flank the mRNA splicing sites of these 658 genes were meticulously designed by a highly reliable primer designing algorithm GENELOOPER™ 2.0 (GeneHarbor Inc) which provides a uniform annealing temperature and PCR product size (62° C. and 300-350 bp, respectively), so that one set of PCR condition can be used for all primer pairs.

Semi-quantitative PCR was conducted in a 20 μl of reaction volume containing 1× reaction buffer, 200 uM dNTPs, 250 nM forward and reverse gene specific primers, 1 unit Taq polymerase (GeneCopoeia Inc, Germantown, Md.) and the cDNAs obtained from uninfected and infected samples in 96-well plates. Typically 46 genes of interest from uninfected and infected samples were analyzed in parallel with one negative control and one GAPDH positive control. The PCR mixture was incubated at 94° C. for 3 min followed by 32 cycles at 94° C. for 30 sec, 62° C. for 30 sec, and 72° C. for 1 min. At the end of PCR amplification, the reaction mixture was held at 72° C. for 5 minutes followed by incubation at 4° C. PCR products from uninfected and infected cDNAs were separated by electrophoresis on 1% agarose gel. Semi-quantitation of the PCR gel image data was performed based on the normalization factor for each cDNA template as described previously. The results show that 22 genes exhibited robust differences in levels of expression in infected vs. uninfected samples, particular at 8 hours post infection. The accession number, names of the genes and their biological processes are listed in Table 1. Not all of these genes showed up-or down-regulation relative to the expression in uninfected sample at all time points. These 22 genes represented 3.3% of the total number of genes identified as 2-fold up-or down-regulated by microarray assays. The protein products of these genes are involved in various biological processes: signal transduction, nucleotide and nucleic acid metabolism, immune response, and one gene each in cell growth and/or maintenance, metabolism and energy pathways, regulation of cell cycle, cell adhesion, and apoptosis according to human protein reference database (www.hprd.org) (29).

TABLE 1
List of genes confirmed by semi-quantitative PCR*.

| Accession | | |
|---|---|---|
| number | Name of Genes | Biological processes |
| NM_000530 | myelin protein zero (Charcot-Maric-Tooth | Signal transduction |
| | neuropathy IB) (MPZ), | |
| NM_000595 | lymphotoxin alpha (TNF superfamily, member | Signal transduction |
| | 1HLTA), | |
| NM_000801 | FK506 binding protein 1A, 12 kDa (FKBP1A), | Signal transduction |
| | transcript variant 12B | |
| NM_001547 | interferon-induced protein with | Cell growth and/or |
| | tetratricopeptide repeats 2 (IFIT2), | maintenance |
| NM 001838 | chemokine (C-C motif) receptor 7 (CCR7), | Signal transduction |
| NM_002498 | NIMA (never in mitosis gene a)-related kinase 3 | Signal transduction |
| | (NEK3), transcript variant 1, | |
| NM 002922 | regulator of G-protein signalling 1 (RGS1), | Signal transduction |
| NM_002946 | replication protein A2, 32 kDa (RPA2), | Nucleotide and nucleic |
| | | acid metabolism |
| NM 002983 | chemokine (C-C motif) ligand 3 (CCL3), | Immune response |
| NM_003205 | transcription factor 12 (HTF4, helix-loop-helix | Nucleotide and nucleic |
| | transcription factors 4) (TCF12), transcript variant 3 | acid metabolism |
| NM_003404 | tyrosine 3-monooxygenase/rryptophan 5- | Signal transduction |
| | monooxygenase activation protein, beta | |
| | polypeptide (YWHAB), transcript variant 1 | |
| NM_003906 | MCM3 minichromosome maintenance deficient 3 | Signal transduction |
| | (S. cerevisiae) associated protein (MCM3AP), | |
| NM_004551 | NADH dehydrogenase (ubiquinone) Fe—S | Metabolism, Energy |
| | protein 3, 30 kDa (NADH-coenzyme Q | pathways |
| | reductase) (NDUFS3), | |
| NM_005082 | tripartite motif-containing 25 (TRIM25), | Nucleotide and nucleic |
| | | acid metabolism |
| NM 005623 | chemokine (C-C motif) ligand 8 (CCL8), | Signal transduction |
| NM_006187 | 2′-5′~oligoadenylate synthetase 3, lOO kDa | Immune response |
| | (OAS3), | |
| NM_007215 | polymerase (DNA directed), gamma 2, | Nucleotide and nucleic |
| | accessory subunit (POLG2), | acid metabolism |
| NM_015369 | TP53TG3 protein (TP53TG3), transcript variant 1 | Regulation of cell |
| | | cycle |
| NM 021991 | junction plakoglobin (JUP), transcript variant 2 | Cell adhesion |
| NM_033340 | caspase 7, apoptosis-related cysteine peptidase | Apoptosis |
| | (CASP7), transcript variant beta | |
| NM_080657 | radical S-adenosyl methionine domain | Immune response |
| | containing 2 (RSAD2), | |
| NM_152998 | enhancer of zeste homolog 2 (Drosophila) | Nucleotide and nucleic |
| | (EZH2), transcript variant 2 | acid metabolism |

*.Gene names are all found in Homo sapiens and the biological processes were assigned based on human protein reference database (HPRD, www.hord.ora). Gene names in bold are those confirmed as down-regulated genes. Genes in bold are down-regulated.

The list of these genes was used to search against the Gene Expression Omnibus (GEO) database in NCBI regardless of the platform of microarray used, the up-or down-regulation of the gene of interest, and the time post infection. The purpose of this investigation was to determine whether the regulation of these genes upon O. tsutsugamushi infection was unique and specific. Each of the 22 genes had been previously identified as regulated by one or several different infectious agents, including virus and bacteria. Some of the infectious agents appeared to simultaneously regulate several of the 22 genes but none of the infectious agents showed regulation of all 22 genes identified in this study. This suggested that the gene expression profile composed of all these 22 genes is O. tsutsugamushi infection specific. Among all the infectious agents searched, A. phagocytophilum induced the regulation of 18 out of 22 genes in promyelocyte cells (NB4). Interestingly, based on the 16S rRNA gene sequence, it is known that both Anaplasma and Orientia along with Rickettsia, Ehrlichia, Neohckettsia and Wolbachia belong to the order Rickettsials. Taken together, the results suggest that even infection by A. phagocytophilum, one of the most closely-related infectious agents to O. tsutsugamushi, can be differentiated from infection by O. tsutsugamushi using the expression profile of these 22 genes.

b. Quantitative PCR Using SYBR Green.

The 22 genes confirmed by semi-quantitative analysis were further examined by SYBR green quantitative real-time PCR. Previous results indicated that most genes showed the greatest difference at 8 hours post infection, thus we decided to focus our analysis on the 8 h post infection samples. The quantitative real time-PCR assays were carried out using the same cDNA templates for semi-quantitative PCR as described previously. SYBR green qPCR experiments were performed in the iCycler (BioRad, Hercules, Calif.) using the light cycler DNA master SYBR green I kit (Roche Diagnostics, Indianapolis, Ind.). The 18S rRNA was used as control (housekeeping gene, HKG) to normalize the raw real-time PCR data of the genes of interest. Sequence information for each primer set is listed in Table 1.

Sensitivity experiments were performed using pBAC-2 cp as a template and plasmid-specific primers designed to produce a 311 bp amplicon. The PCR was initiated with a 2-minute denaturation at 95° C. followed by 40 cycles at 95° C. for 15 seconds, 20 seconds annealing at 60° C., and 30 seconds extension at 72° C. After the completion of 40 cycles, the reaction mixture was held at 72° C. for 5 min followed by incubation at 4° C. The standard curve had a serial 1:10 dilution of DNA template starting from 1 ng down to 100 fg. One additional ten-fold dilution was made after the standard was undetectable. The analyses of data were accomplished using the iCycler Software.

Serial dilutions were used to determine the efficiency (E) of each primer set according to the following equation: E=(10⁻¹ m)⁻¹ where m represents the slope of the best fitted straight line of the graph of Ct (threshold cycle) vs. the corresponding range of dilution factors of cDNA.

The Ct values for all the genes were converted to a fold change using the formula [(1+E) ΔCt]GOI/[1+E) ΔCt]HKG, where ΔCt denotes the difference between the Ct values of uninfected and infected samples of a given gene. GOI and HKG symbolizes genes of interest and housekeeping genes (18S rRNA) respectively.

All 22 genes showed differences between the control and infected samples (data not shown) but the differences were most prominent for NM_{—}001547 (IFIT2) and NM_{—}006187 (0AD3), known to be involved in cell growth and/or maintenance and immune response, respectively. Similar results for these two genes were observed for samples from different time points although the magnitude of differences was lower than those observed from 8 hours post infection samples (data not shown).

TABLE 2
Primers used in SYBR Green quantitative PCR^{a}

| Primer ID | Oligonucleotide sequence |
|---|---|
| 18S-F | S′-CTCGATGCTCTTAGCTGAGTGTC\hat{ }′ |
| 18S-R | 5′-GAACGCCACTTGTCCCTCTAAG-3′ |
| KMJ)00530-F | 5′-CAATGGCACGTTCACTTGTGACG-3′ |
| NM_000530-R | 5′-CTTCTC ACTGACAGCTTTGGTGC-3′ |
| NM_000595-F | 5′-ATCTTGCCCACAGCACCCTCAAAC-3′ |
| NM_000595-R | 5′-CAGCCCTGGATACACCATCTTCTG-3′ |
| NM_000801-F | 5′-ATGCTAGGCAAGCAGGAGGTGAT-3′ |
| NM_000801-R | 5′-GA A AC AGAGGTGTCGGAAGCAAAG-3* |
| NM_001547-F | 5′-A ATAGG ACACGCTGTGGCTCATC-3′ |
| NM_001547-R | 5′-CTCCTGAAGGAATGCCAAGACATG-3′ |
| NM_001838-F | 5′-CC A ATG AA A AGCGTGCTGGTGGT-3′ |
| NM_001838-R | 5′-AAAGTGGACACCGAAGACCCAGG-3′ |
| NM_002498-F | S′-GTCAGTCCATCTGAGGAAAGCCA\hat{ }′ |
| NM_002498-R | 5′-TGACCTCCATCAACACTGTCCGA-3′ |
| NM_002922-F | 5′-GAGTTCTGGCTGGCTTGTGAAGAC-3′ |
| NM_002922-R | 5′-GGAGCCATACTGGCACATTCCTTC-3′ |
| NM_002946-F | 5′-ATGACAGCTGCACCCATGGACG-3′ |
| NM_002946-R | S′-CCTTCAGGTCTTGGACAAGCCTT-S′ |
| NM_02983-F | 5′-GGTGTCATCTTCCTAACCAAGCG-3′ |
| NM_002983-R | 5′-GCTGATGACAGCCACTCGGTTG-3′ |
| NM_003205-F | 5′-TCTCCTGACCATACCAGCAGTAG-3′ |
| NM_003205-R | 5′-AGACTG ACAG AGTCTTCCCGATG-3′ |
| NM_003404-F | 5′-TCGGCTGTGGATAGAGAAGCAGG-3^{>} |
| NM_003404-R | 5′-CACCTTACTTTCTGGTTGTGTAGC-3′ |
| NM_003906-F | 5′-CAGTTCCTGGCTTCTGTGGTGTC-3′ |
| NM_003906-R | 5′-CTTGCTCTTCCACCTACAGTAGG-3′ |
| NM_004551-F | 5′-CAACCTGTTGTCTCTGCGCTTCA-3* |
| NM_004551-R | 5′-TTGGCGATAGACTGGGAAAGCCT-3′ |
| NM_005082-F | 5′-CC AAGTCC AGACCTGAGCTCCT-3′ |
| NM_005082-R | S′-GTGGTCACAGTTGAGAAGCACGC-o′ |
| NM_005623-F | 5′-CAAGGAGAGATGGGTCAGGGATT-3′ |
| NM_005623-R | 5′-CCCACAACACTACAGACAGGTAG-3′ |
| NM_006187-F | 5′-TGGCTCTTCAGCCAAAGGCACAG-3′ |
| NM_006187-R | 5′-GCTCTGTGAAGCAGGTGGAGTAC-3′ |
| NM_007215-F | 5′-UdTCACGGTGCCCTGGAACAC-3′ |
| NM_007215-R | 5′-CGTGATCTCCTAAGTTCCACAGG-3′ |
| NM_015369-F | 5′-CG ATTTCCTGTCAGCCAACA AAGG-3′ |
| NM_015369-R | S′-TCTGTCTCTTCCCGCTTTTCCTC-3′ |
| NM_021991-F | S′-CTGGTGCAGAACTGCCTGTGGA-3′ |
| NM_021991-R | 5′-GGATGCCATAGTTGAGACGCACA-3′ |
| NM_033340-F | S′-GTGATCTCGGAAGACTGCAACCT-3* |
| NM_033340-R | 5′-AGAGTTCCTTGGTGAGCATGGAG-3′ |
| NM_080657-F | 5′-CGCCACAAAGAAGTGTCCTGCTTG-V |
| NM_080657-R | 5′-GACCACAGGTAATCAGATGCCACG-3′ |
| NMJ52998-F | 5′-TGTGG AGTTGGTGAATGCCCTTG-3′ |
| NMJ52998-R | 5′-ACATCGCCTACAGAAAAGCGTATG-3′ |

c. Fluorgenic Probes for Quantitative Real Rime PCR

Both NM_{—}001547 and NM_{—}006187 were further evaluated using quantitative real time PCR with primers and probes designed specifically for the two genes. The assay was formulated to employ a uniquely designed internal fluorescence-labeled probe complementary to a target sequence of each amplicon using a pair of flanking primers. ORF regions of the two genes (NM_{—}001547 and NM_{—}006187) were analyzed to develop probes for TaqMan quantitative PCR. The primer pairs and probes of NM_{—}001547 and NM_{—}006187 genes for real time PCR were designed using Primer Express software (PE Applied Biosystem Inc., Foster City, Calif.) and are shown in Table 3.

TABLE 3
Primers and probes used in quantitative
real-time PCR^{a}

| Primer ID | Oligonucleotide sequence |
|---|---|
| 18S-F | 5′-CTTTCGATGGTAGTCGCCGT-3′ |
| 18S-R | 5′-TTGGAGCTGG AATTACCGCG-3′ |
| 18S-P | 5′FAM-CCACATCCAAGGAAGGCAGC |
| | AGGC-3′TAMRA |
| NM_001547-F | 5′-TTCACCTCTGGACTGGCAA-3′ |
| NM_001547-R | 5′-TTCAGAGCCAGGAGGACTT-3′ |
| NM_001547-P | 5′FAM-CCATTGACCCTCTGAGGCAA |
| | GCCA-3′TAMRA |
| NM_006187-F | 5′-GCTTTCTGAGTAGAGACGG-3′ |
| NM_006187-R | 5′-CACTGATGAACTTGTCAAGG-3′ |
| NM 006187-P | 5′FAM-ATGTGATGCCAGCCCTCCTT |
| | TACCAAA-3′TAMRA |

^{a}Genes follow by F, R and P represent forward primer, reverse primer and probe used for the experiment.

The fluorescence labeled oligonucleotide probe was labeled with 5′-FAM (reporter dye) and 3′-TAMRA dyes (quencher dye). After hybridizing to the target amplicon, fluorescent signal was generated by separating the reporter dye from the quencher dye through 5′-nuclease activity of DNA Taq polymerase. Labeled probes were synthesized by Eurogentec (San Diego, Calif.) and unlabeled primer pairs were synthesized by MWG Biotech Inc. (High Point, N.C.). PCR was conducted in 50 μl reaction volumes containing 2 μl cDNA template, 5 μl 10× Taqman buffer (PE ABI), 4 μl 1.25 mM dNTPs, 8 μl 1.25 mM MgCl₂, 200 mM forward and reverse primers, 20 nM fluoregenic probe, and 1.25 units AmpliTaq Gold DNA polymerase (PE ABI). A gene detection system from MJ research (DNA Engine Opticon 2 Real-Time Cycler) was employed for PCR cycling amplification, real time data.

PCR mixtures were pre-incubated at 50° C. for 2 min, then 95° C. for 10 min followed by 40 cycles of two-step amplification at 95° C. for 15 second and 60° C. for 1 min. The 4, 8, and 18 hours post infection infected and uninfected samples from one of the three donors were analyzed.

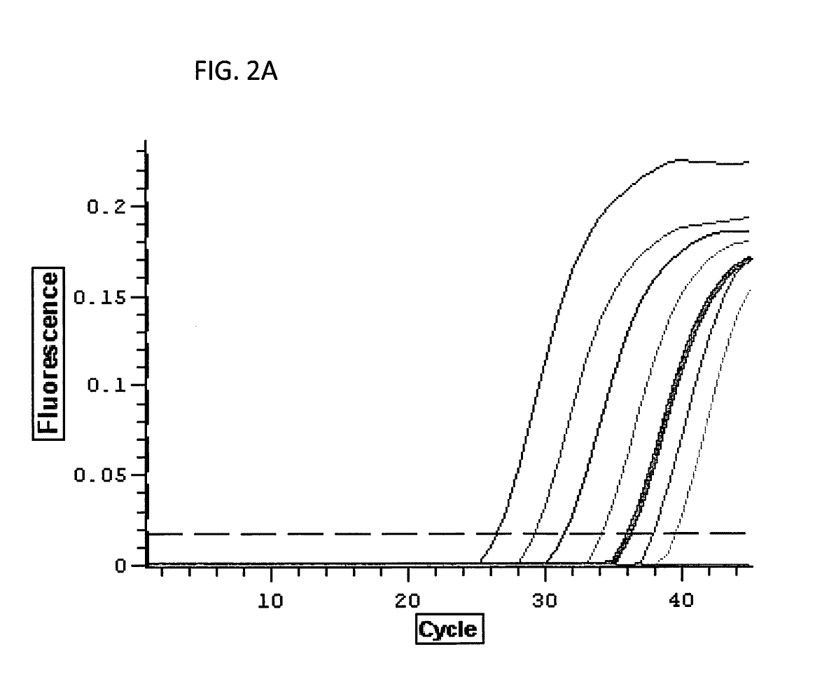

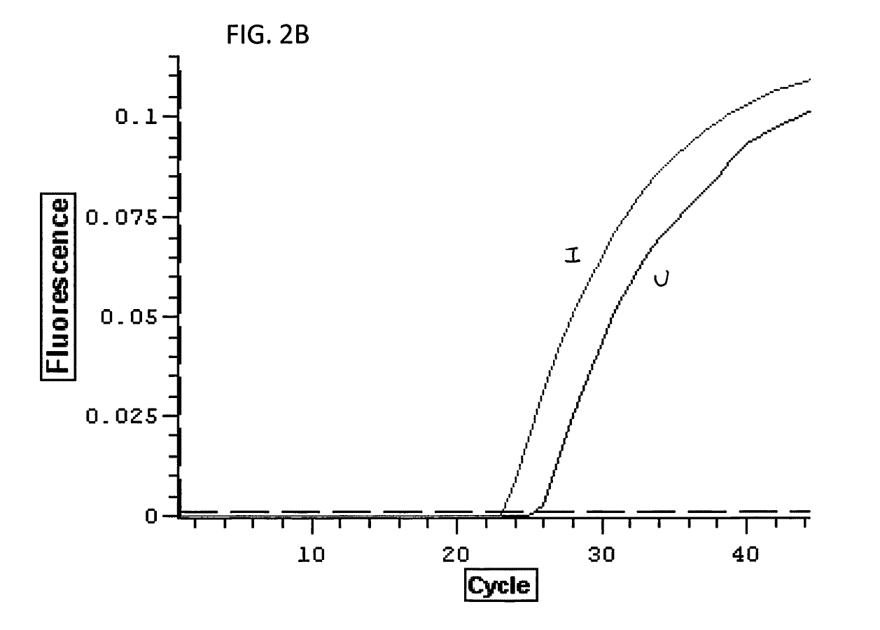

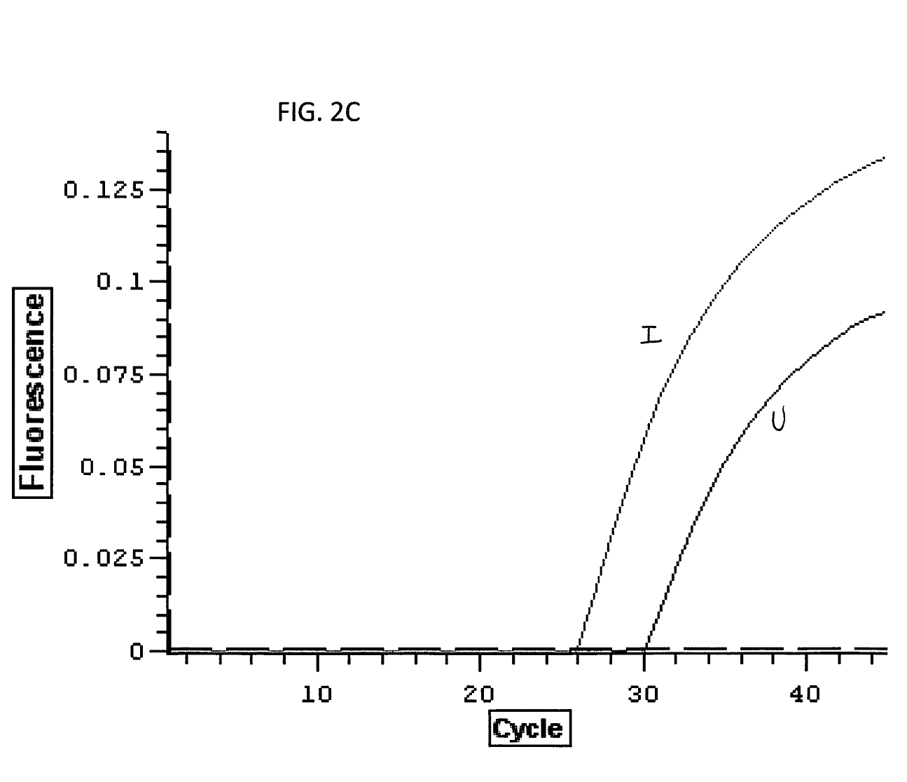

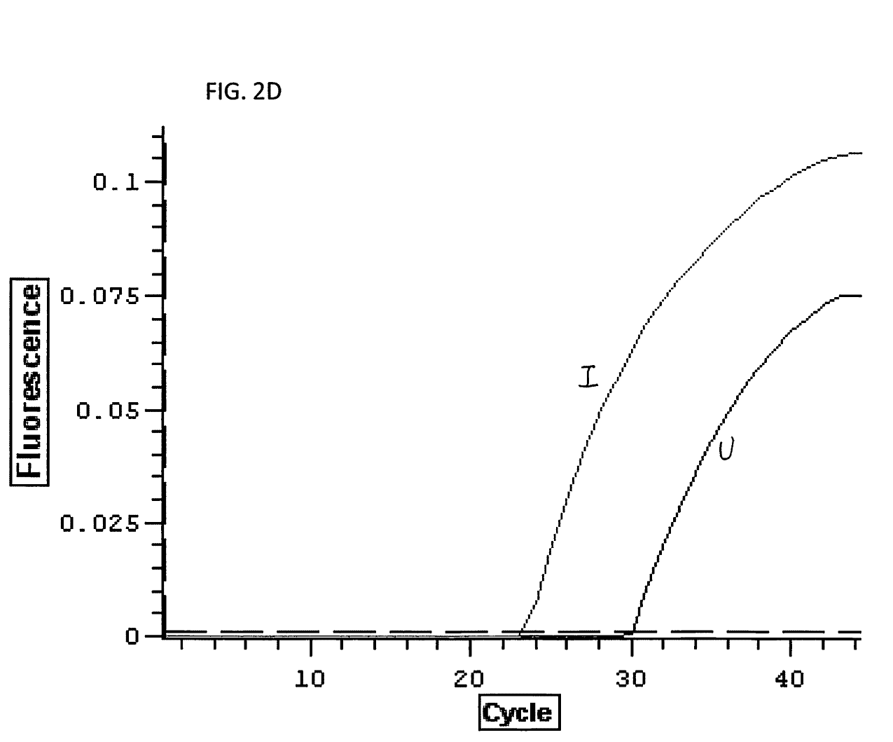

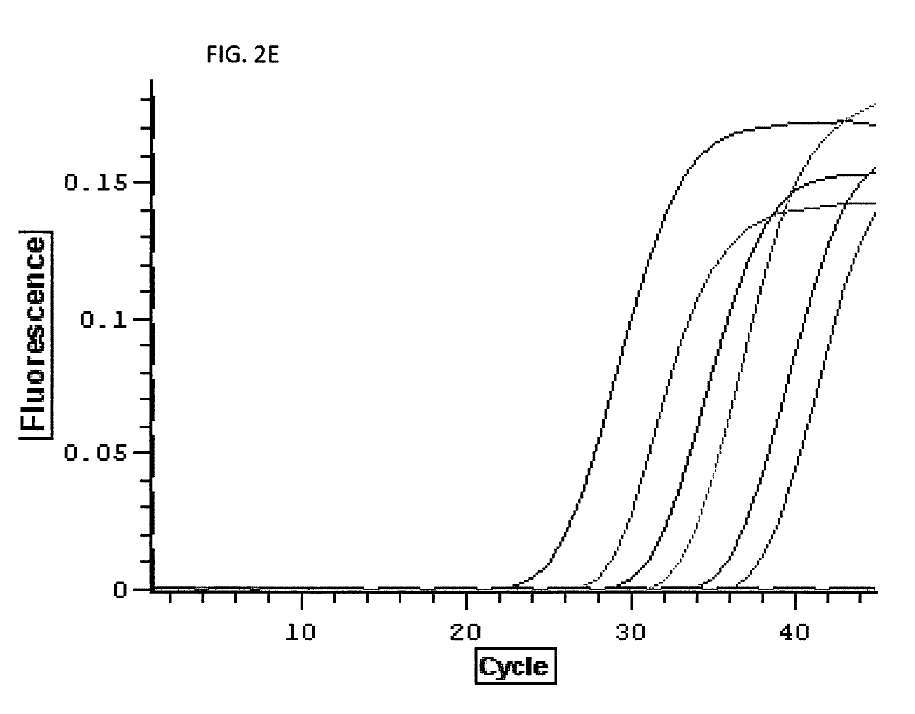

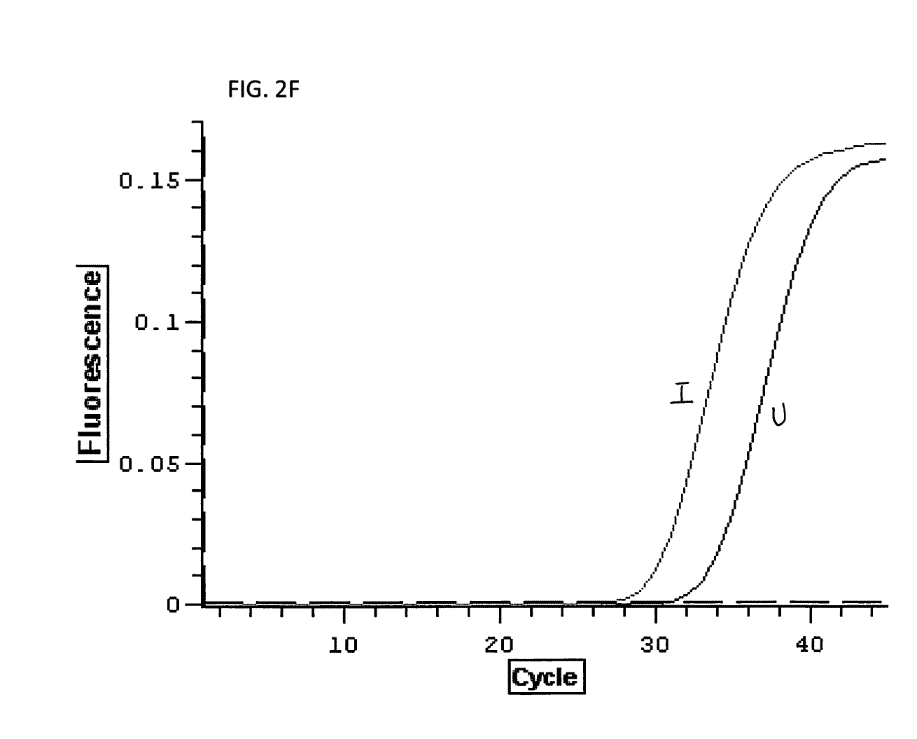

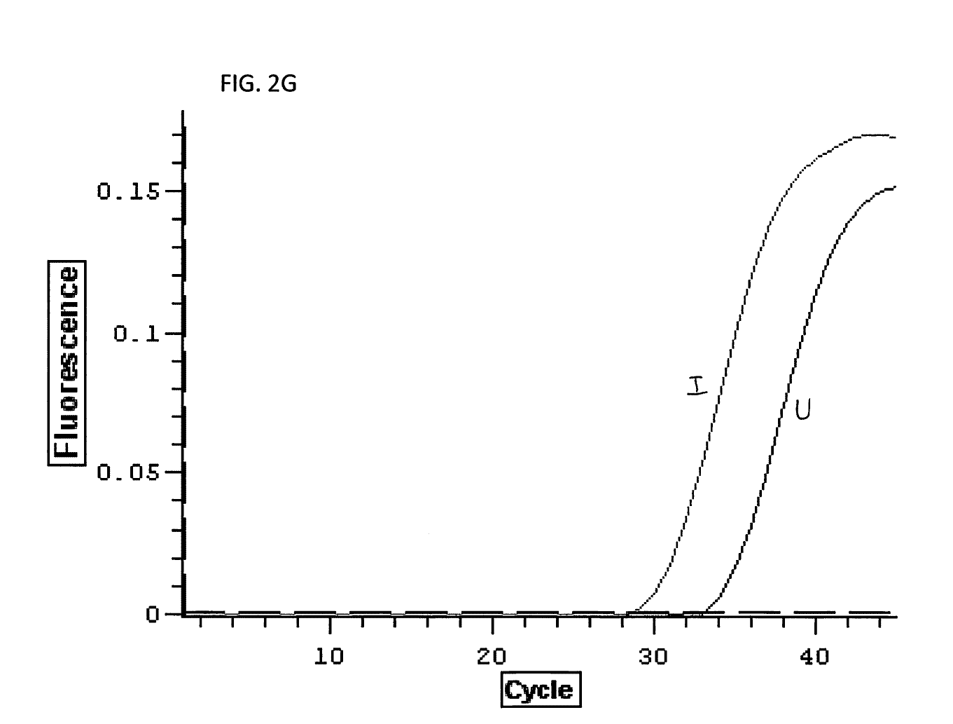

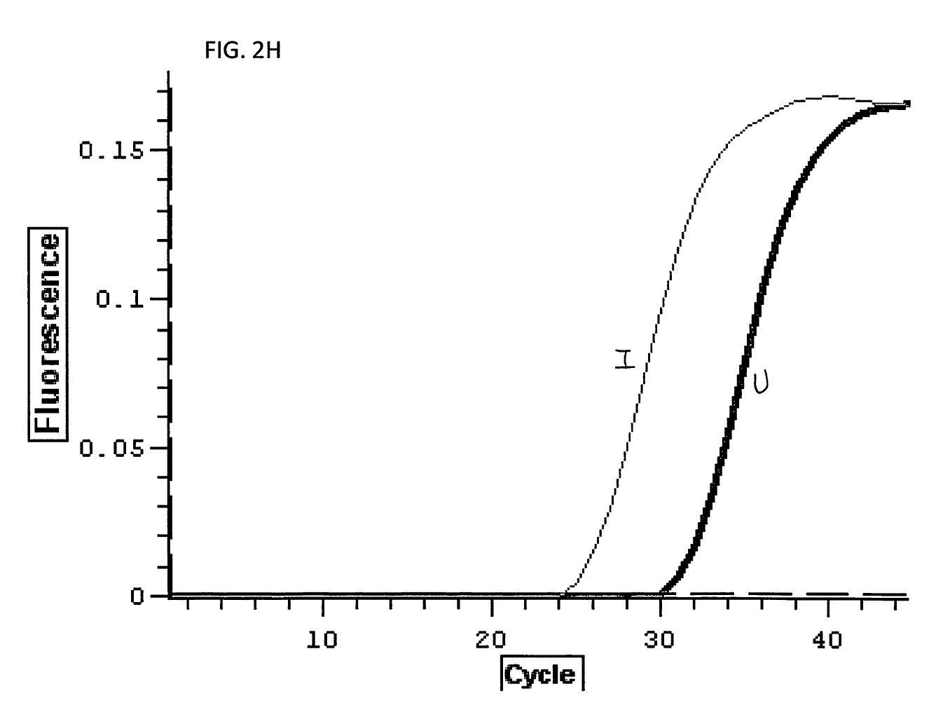

The results are shown in FIG. 2 (similar results were observed with the other donors, data not shown). In panels A through D, the results of NM_{—}001547 are shown and panels E through H show the results of NM_{—}006187. The insets in panels B to D and F to H showed that the amounts of cDNA for the 18S rRNA were equivalents for both control and infected samples, indicating that the differences in the cycle number for both target genes in each sample are due to differences in the number of their transcripts (cDNA copies). Thus both NM_{—}001547 and NM_{—}006187 are up-regulated in infected samples and the up-regulation is persistent for at least 18 hours post infection. Table 3 shows the results of the expression of both NM_{—}001547 and NM_{—}006187 in PBMCs infected by different infectious agents 8 hours post infection. The up-regulation of both genes were not observed in the infection of NB4 by A. phagocytophilum, indicating that one may be able to use just these two genes to differentiate O. tsutsugamushi infection from A. phagocytophilum. However, only virus infection did not result in the up-regulation of both genes whereas bacterial infection or exposure to toxin led to the up regulation of both genes. Therefore, the change of expression of these two genes alone is not sufficient to differentiate various bacterial infections. The up-regulation of these 2 genes was also observed at 18 h post infection, suggesting that up-regulation was a sustained effect for at least 18 h post infection. Some of the 22 genes were similarly regulated at 18 h post infection as they were at 8 h post infection. Therefore, the 8 h post infection in vitro is probably the optimal time to obtain samples for differentiating O. tsutsugamushi infection from other infections. It is thus plausible to construct a diagnostic platform based on these 22 genes along with necessary control genes to monitor the expression of these genes. Recent advancement in microarray technology makes it possible to perform expression profiling experiment with complete data analysis within 1 day, making DNA microarray analysis an attractive method to effectively diagnose early Orientia infection (8-18 hours post infection).

TABLE 3
Fold Change of Gene Expression from
PBMC Infected by Various Agents

| | |
|---|---|
| Pathogen or Toxin | Fold Change |

| Used* | NM_001547 | NM_00618 |
|---|---|---|
| Anthrax infection | 5.3 | 5.4 |
| VEE | 0.48 | 0.85 |
| SEB | 5.5 | 2.7 |
| BOT | 23.5 | 18.4 |
| Dengue | 0.86 | 2.7 |
| Rickettsia | 9.4 | 8.8 |

*VEE: Venezuelan Equine encephalitis virus, SEB: Staphylococcal enterotoxin B, BOT: C. botulinum toxin.

### REFERENCES

1. Brown, G. W., D. M. Robinson, D. L. Huxsoll, T. S. Ng, K. J. Lim and G. Sannasey. 1976. Scrub typhus: a common cause of illness in indigenous populations. Trans. R. Soc. Trop. Med. Hyg. 70:444-448.
2. Brown, G. W., J. P. Saunders, S. Singh, D. L. Huxsoll, and A. Shirai. 1978. Single dose doxycycline therapy for scrub typhus. Trans. R. Soc. Trop. Med. Hyg. 72: 412-416.
3. Bozemean, F. M., And B. L. Elisberg. 1963. Serological diagnosis of scrub typhus by indirect immunofluorescence. Proc. Soc. Exp. Biol. Med. 112:568-573.
4. Dasch, G. A., S. Halle, and A. L. Bourgeois. 1979. Sensitive microplate enzyme-linked immunosorbent assay for detection of antibodies against the scrub typhus rickettsia, Rickettsia tsutsugamushi. J. clin. Microbiol. 9:38-48.
5. Dohany, A. L., A. Shirai, D. M. Robinson, S. Ram, and D. L. Huxsoll. 1978. Identification and antigenic typing of Rickettsia tsutsugamushi in naturally infected chiggers (Acarina: Trombiculidae) by direct immunofluorescence. Am. J. Trop. Med. Hyg. 27:1261-1264.
6. Kelly, D. J., P. W. Wong, E. Gan and G. E. Lewis, Jr. 1988. Comparative evaluation of the indirect immunoperoxidase test for the serodiagnosis of rickettsial disease. Am. J. Trop. Med. Hyg. 38:400-406.
7. Suto, T. 1980. Rapid serological diagnosis of tsutsugamushi disease employing the immuno-peroxidase reaction with cell cultured ricketsia. Clin. Viol. 8:425-429.
8. Suwanabun, N., C. Chouriyagune, C. Eamisila, P. Watcharapichat, G. A. Dasch, R. S. Howard and D. J. Kelly. 1997. Evaluation of an enzyme-linked immunosorbet assay in Thai scrub typhus patients. Am. J. Trop. Med. Hyg. 56:38-43.
9. Weddle, J. R., T. C. Chan, K. Thompson, H. Paxton, D. J. Kelly, G. Dasch, and D. Strickman. 1995. Effectiveness of a dot-blot immunoassay of anti-Rickettsia tsutsugamushi antibodies for serologic analysis of scrub typhus. Am. J. Trop. Med. Hyg. 53:43-46.
10. Yamamoto, S., and Y. Minamishima. 1982. Serodiagnosis of tsutsugamushi fever (scrub typhus) by the indirect immunoperoxidase technique. J. Clin. Microbiol. 15:1128-1132.
11. Murata, M. Y. Yoshida, Osono, N. Ohashi, Oyanagi, H. Urakami, A. Tamura, S. Nogami, H. Tanaka, and A. Kawamura, Jr. 1986. Production and characterization of monoclonal strain-specific antibodies against prototype strains of Rickettsia tsutsugamushi. Microbiol. Immunol. 30:599-610.
12. Furuya, Y., Y. Yoshida, T. Katayama, F. Kawamori, S. Yamamoto, N. Ohashi, A. Kamura and A. Kawamura, Jr. 1991. Specific amplification of Rickettsia tsutsugamushi DNA from clinical specimen by polymerase chain reaction. J. Clin. Microbiol. 29:2628-2630.
13. Kelly, D. J., G. A. Dasch, T. C. Chye, and T. M. Ho. 1994. Detection and characterization of Rickettsia tsutsugamushi (Rickettsiales: Rickettsiaceae) in infected Leptotrombidium (Leptotrombidium) fletcheri chiggers (Acari: Trombiculidae) with the polymerase chain reaction. J. Med. Entomol. 31:691-699.
14. Dasch, G. A., D. Strickman, G. Watt, and C. Eamsila. 1996. Measuring genetic variability in Orientia tsutsugamushi by PCR/RFLP analysis: a new approach to questions about its epidemiology, evolution and ecology, p. 79-84. In J. Kazar (ed.) Rickettsiae and Rickettsial Diseases. Vth International Symposium. Slovak Academy of Sciences, Bratislava.
15. Enatsu, T., H. Urakami, A. Tamura. 1999. Phylogenetic analysis of Orientia tsutsugamushi strains based on the sequence homologies of 56-kDa type-specific antigen genes. FEMS 180:163-169.
16. Horinouchi, H., K. Murai, A. Okayama, Y. Nagatomo, N. Tachibana, and H. Tsubouchi. 1996. Genotypic identification of Rickettsia tsutsugamushi by restriction fragment length polymorphism analysis of DNA amplified by polymerase chain reaction. Am. J. Trop. Med. Hyg. 54:647-651.
17. Ohashi, N., Y. Koyama, H. Urakami, M. Fukuhara, A. Tamura, F. Kawamori, S. Yamamoto, S. Kasuya, and K. Yoshimura. 1996. Demonstration of antigenic and genotypic variation in Orientia tsutsugamushi which were isolated in Japan, and their classification into type and subtype. Microbiol. Immunol. 40:627-638.
18. Tamura, A., N. Ohashi, Y. Koyama, M. Fukuhara, F. Kawamori, M. Otsuru, P-F. Wu, and S-Y. Lin. 1997. Characterization of Orientia tsutsugamushi isolated in Taiwan by immunofluoescence and restriction fragment length polymorphism analyses. FEMS Microbiol. Lett. 150:225-231.
19. Jett, M. Alterations in Gene Expression Show Unique Patterns in Response to Toxic Agents. 21st Army Science Conference, Proceedings 21:529-534.
20. Nau, G. J., J. F. L. Richmon, A. Schlesinger, E. G. Jennings, E. S. Lander and R. A. Young. 2002. Human macrophage activation programs induced by bacterial pathogens. Proc. Natl. Acad. Sci (USA) vol 99 (3):1503-1508.
21. Lockhart D J and E. A. Winzeler. 2000. Genomics, gene expression and DNA arrays. Nature. 405:827-836.
22. Eckmann L, J. R. Smith, M. P. Housley, M. B. Dwinell and M. F. Kagnoff. 2000. Analysis by high density cDNA arrays of altered gene expression in human intestinal epithelial cells in response to infection with the invasive enteric bacteria Salmonella. J. Biol. Chem. 275(19):14084-14094.
23. Blader I J, I. D. Manger, and J. C. Boothroyd. 2001. Microarray analysis reveals previously unknown changes in Toxoplasma gondii-infected human cells. J. Biol. Chem. 276:24223-24231.
24. Ge, H., Y.-Y. Chuang, E., S. Zhao, J. J. Temenak, W.-M. Ching, W.-M. 2004. Comparative genomics of Rickettsia prowazekii Madrid E and Breinl strains. J. Bacteriol 186 (2):556-565.
25. Yang, Y. H., Dudoit, S., Luu, P., Lin, D. M., Peng, V., Ngai, J. and Speed, T. P. 2002. Normalization for cDNA microarray data: A robust composite method addressing single and multiple slide systematic variation. Nucleic Acids Research 30:e15.
26. Jiang J, Chan T C, Temenak J J, et al. Development of a quantitative real-time polymerase chain reaction assay specific for Orientia tsutsugamushi. Am J Trop Med Hyg 2004; 70:351-6.
27. Das R, Hammamieh R, Neill R, et al. Early indications of exposure to biological threat agents using host gene profiles in peripheral blood mononuclear cells. BMC Infect Dis 2008; 8: 104.
28. Imbeaud S, Graudens E, Boulanger V, et al. Towards standardization of RNA quality assessment using user-independent classifiers for microcapillary electrophoresis traces. Nucleic Acid Res 2005; 33: e56-e67.
29. Peri S. Navarro J D, Amanchy R, et al. Development of human protein reference database as an initial platform for approaching systems biology in humans. Genom Res 2003; 13:2363-71.

Having described the invention, one of skill in the art will appreciate in the claims that many modifications and variations of the present invention are possible in light of the above teachings. It is therefore, to be understood that, within the scope of the claims, the invention may be practiced otherwise than as specifically described.

What is claimed is:

1. A method for the early diagnosis of Orientia tsutsugamushi infection wherein diagnosis is by determining a gene expression profile comprising the steps:
a. obtaining total RNA from cells from a patient, total RNA from uninfected control cells and RNA from cells infected with Orientia tsutsugamushi;
b. measuring the expression of genes from said patient cells, uninfected control cells and Orientia tsutsugamushi infected cells to obtain gene expression profile comprising the genes; lymphotoxin alpha, FK506 binding protein, interferon induced protein with tetratricopeptide repeats 2, chemokine receptor 7, never-in-mitosis gene a-related kinase 3, chemokine ligand 3, transcription factor 12, minichromosome maintenance deficient 3 associated protein, NADH dehydrogenase Fe—S protein 3, chemokine ligand 8,2′-5′ oligoadenylate synthetase 3, junction plakoglobin, replication protein A2, G protein signaling 1, apoptosis-related cysteine protease, tyrosine 3-monooxygenase/tryptophan 5-monooxygenase activation protein, beta polypeptide, polymerase gamma 2 accessory subunit, enhancer of zeste homology 2, tripartite motif-containing 25, radical S-adenosyl methionine domain and to the expected gene profile of genes expected to be repressed including: myelin protein zero, TP inducible gene;
c. determining the modulation of said expression of said genes by comparing the expression of said patient genes with the expression of said genes from said infected and uninfected cells;
d. creating a profile of the modulation of said patient, infected and uninfected cell genes;
e. comparing said patient cell gene modulation profile to the profile to the profile of said infected and uninfected cell genes.

2. The method of claim 1, wherein said infected, uninfected and patient cells are selected from the group consisting of leukocytes, peripheral blood lymphocytes and mononuclear cells.

3. The method of claim 2, wherein said measurement of gene expression is by microarray analysis comprising the steps:
a. synthesizing a cDNA copy of said RNA with a labeled;
b. hybridizing said labeled cDNA to DNA sequences immobilized on microarray chips encoding said genes expected to be induced and repressed following Orientia tsutsugamushi infection;
c. measuring the amount of hybridization of said labeled cDNA to obtain said patient gene profile;
d. comparing said patient gene profile to said expected gene profile.

4. The method of claim 3 wherein said label is a fluore selected from the group consisting essentially of Cy3 and Cy5.

5. The method of claim 2, wherein said measuring of expression is by reverse transcriptase polymerase chain reaction.

6. The method of claim 5 wherein the primers sets for said reverse transcriptase polymerase chain reaction contain a primer complementary to the sequence encoding the splice site of the target mRNA.

7. The method of claim 6, wherein said reverse transcriptase polymerase chain reaction comprising the steps:
a. synthesizing a cDNA copy of said RNA;
b. amplifying said cDNA by polymerase chain reaction using forward and reverse primers to a control house-keeping gene and to one or more genes including: lymphotoxin alpha (LTA), FK506 binding protein (FKBP1A), interferon induced protein with tetratricopeptide repeats 2 (IFIT2), chemokine receptor 7 (CCR7), never-in-mitosis gene a-related kinase 3 (NEK3), chemokine ligand 3 (CCL3), transcription factor 12 (TCF12), minichromosome maintenance deficient 3 associated protein (MCM3AP), NADH dehydrogenase Fe—S protein 3 (NDUFS3), radical S-adenosyl methionine domain (RSAP2), chemokine ligand 8 (CCL8), 2′-5′ oligoadenylate synthetase 3 (OAS3), junction plakoglobin (JUP), tripartite motif-containing 25 (TRIM25), replication protein A2 (RPA2), G protein signaling 1 (RGS1), apoptosis-related cysteine protease (CASP7), tyrosine 3-monooxygenase/tryptophan 5-monooxygenase activation protein, beta polypeptide (YWHAB), polymerase gamma 2 accessory subunit (POLG2), enhancer of zeste homology 2 (EZH2), myelin protein zero (MPZ) and TP inducible (TP53TG3);
c. separating polymerase chain reaction products by gel electrophoresis;
d. measuring the relative expression of said electrophoresis separated products.

8. The method of claim 6, wherein said reverse transcriptase polymerase chain reaction is real-time reverse transcriptase polymerase chain reaction comprising the steps:
a. synthesizing a reporter dye and quencher dye labeled cDNA copy of said RNA;
b. amplifying said cDNA using forward and reverse primers specific to a control gene and one or more genes including: lymphotoxin alpha, FK506 binding protein, interferon induced protein with tetratripeptide repeats 2, chemokine receptor 7, never-in-mitosis gene a-related kinase 3, chemokine ligand 3, transcription factor 12, minichromosome maintenance deficient 3 associated protein, NADH dehydrogenase Fe—S protein 3, chemokine ligand 8,2′-5′ oligoadenylate synthetase 3, junction plakoglobin, replication protein A2, G protein signaling 1, apoptosis-related cycteine protease, tyrosine 3-monooxygenase/tryptophan 5-monooxygenase activation protein, beta polypeptide, polymerase gamma 2 accessory subunit, enhancer of zeste homology 2, tripartite motif-containing 25, radical S-adenosyl methionine domain and to the expected gene profile of genes expected to be repressed including: myelin protein zero, TP inducible gene;
c. determining the number of polymerase chain reaction cycles required for detection of said reporter dye;
d. comparing said number of polymerase chain reaction cycles required for detection between said patient RNA, RNA from infected and RNA from uninfected cells.

9. The method of claim 8, wherein said reporter dye is 5′-FAM and said quencher dye is 3′-TAMRA.

10. The method of claim 2, wherein said measurement of gene expression is by enzyme-linked immunosorbent assay comprising the steps:
a. extracting total protein from said cells;
b. immobilizing specific quantities of said total protein and exposing each of said immobilized quantity of total protein to an antibody specific for a house keeping gene and one or more of the genes including: lymphotoxin alpha (LTA), FK506 binding protein (FKBP1A), interferon induced protein with tetratricopeptide repeats 2 (IFIT2), chemokine receptor 7 (CCR7), never-in-mitosis gene a-related kinase 3 (NEK3), chemokine ligand 3 (CCL3), transcription factor 12 (TCF12), minichromosome maintenance deficient 3 associated protein (MCM3AP), NADH dehydrogenase Fe—S protein 3 (NDUFS3), radical S-adenosyl methionine domain (RSAP2), chemokine ligand 8 (CCL8), 2′-5′ oligoadenylate synthetase 3 (OAS3), junction plakoglobin (JUP), tripartite motif-containing 25 (TRIM25), replication protein A2 (RPA2), G protein signaling 1 (RGS1), apoptosis-related cysteine protease (CASP7), tyrosine 3-monooxygenase/tryptophan 5-monooxygenase activation protein, beta polypeptide (YWHAB), polymerase gamma 2 accessory subunit (POLG2), enhancer of zeste homology 2 (EZH2), myelin protein zero (MPZ) and TP inducible (TP53TG3);
c. measuring the relative expression of said genes by measuring the binding of specific antibody to said gene product and said house-keeping gene product.

11. The method of claim 1, wherein the early diagnosis of Orientia tsutsugamushi infection occur at 8-18 hours post infection.
